(19) United States
(12) Patent Application Publication
(10) Pub. No.: US 2016/0375261 A1
(43) Pub. Date: Dec. 29, 2016

(54) Event-Driven Transmission of Treatment Data

(71) Applicant: Physio-Control, Inc., Redmond, WA (US)

(72) Inventors: Moira M. Galvin, Kirkland, WA (US); Kevin C. Drew, Snohomish, WA (US); Dana S. Lewis, Woodinville, WA (US); Jennifer Krebsbach, Redmond, WA (US); Denise Norman, Woodinville, WA (US); Melissa Pochop-Miller, Seattle, WA (US); Steven B. Duke, Edmonds, WA (US); Mark Killebrew, Woodinville, WA (US)

(21) Appl. No.: 15/194,485

(22) Filed: Jun. 27, 2016

Related U.S. Application Data
(60) Provisional application No. 62/184,801, filed on Jun. 25, 2015.

(51) Int. Cl. A61N 1/372; A61N 1/39

(52) U.S. Cl. CPC A61N 1/37282; A61N 1/3925

(57) ABSTRACT

An event-driven medical treatment data notification system is disclosed. Embodiments are directed to a system for transmitting medical treatment data, such as AED device events, waveforms, and device location information, to recipients that have registered to receive the data. In specific embodiments, the data is recorded by medical devices in the course of treatment during a medical incident. In specific embodiments, recipients register to receive treatment data in response to certain events. The treatment data is transmitted to any registered recipients in response to the occurrence of certain events.

### CROSS REFERENCE TO RELATED APPLICATIONS

This patent application claims the benefit of and priority to U.S. Provisional Patent Application Ser. No. 62/184,801 filed on Jun. 25, 2015, entitled “Medical Device Event Viewer,” the disclosure of which is hereby incorporated by reference for all purposes.

### TECHNICAL FIELD

The disclosed subject matter pertains generally to the area of medical devices, and more specifically to the area of exposing medical data collected at the point-of-treatment to fast responders.

### BACKGROUND INFORMATION

Health care providers often deploy medical device systems to facilitate the provision of health care services. Portable medical devices, such as defibrillators and mechanical compression devices, are frequently used in the field to help in the treatment of patients experiencing a medical incident. For example, portable defibrillators are often deployed at locations in the field so that they may be quickly accessed by individuals with medical training to help treat patients experiencing a sudden cardiac event, such as sudden cardiac arrest or a heart attack.

A critical element of effective care for such a medical incident is timely notice of the type of problem and the treatment being administered to the patient. Existing medical devices are known that include the capability to record data about treatment being provided, including measurements of the patient's condition made by the medical devices during the treatment.

A superior status monitoring ability for use in a medical device system has eluded those skilled in the art, until now.

### SUMMARY OF EMBODIMENTS

Embodiments are directed to a system for transmitting medical treatment data, such as AED device events, waveforms, and device location information, to recipients that have registered to receive the data. In specific embodiments, the data is recorded by medical devices in the course of treatment during a medical incident. In specific embodiments, recipients register to receive treatment data in response to certain events. The treatment data is transmitted to any registered recipients in response to the occurrence of certain events.

### BRIEF DESCRIPTION OF THE DRAWINGS

FIG. 1 is a diagram of a scene where an external defibrillator is used to try and save the life of a person in accordance with an embodiment.

FIG. 2 is a table listing two main types of the external defibrillator shown in FIG. 1, and by whom they might be used.

FIG. 3 is a diagram showing components of an external defibrillator made according to embodiments.

FIG. 4 is a functional block diagram generally illustrating a medical device system in accordance with one embodiment.

FIG. 5 is a conceptual illustration of an event-driven system for transmitting treatment data to registered recipients in accordance with one embodiment.

FIG. 6 is a functional flow diagram generally illustrating one embodiment of a process performed by an illustrative system for transmitting treatment data.

FIG. 7 is a functional flow diagram generally illustrating one embodiment of a process performed by an illustrative system for registering recipients to receive treatment data based on events for which that recipient is interested.

FIG. 8 is a functional block diagram generally illustrating one possible example of a computing device that may be used in various embodiments.

### DETAILED DESCRIPTION

Generally described, the disclosure is directed to a medical device system for transmitting medical treatment data to recipients that have registered to receive the data. In specific embodiments, the data is recorded by medical devices in the course of treatment during a medical incident. In specific embodiments, recipients register to receive treatment data in response to certain events. The treatment data is transmitted to any registered recipients in response to the occurrence of certain events. Recipients may include, for example, medical personnel who may be or become involved in the treatment of the patient. The treatment data may include, for example, defibrillator device events, waveforms, device location information, and the like.

This disclosure will begin with a description of one example of a medical device that may be used in specific embodiments. Next is a discussion of one specific example of an event-based system for distributing treatment data to registered recipients.
Description of Operative Environment for Embodiments

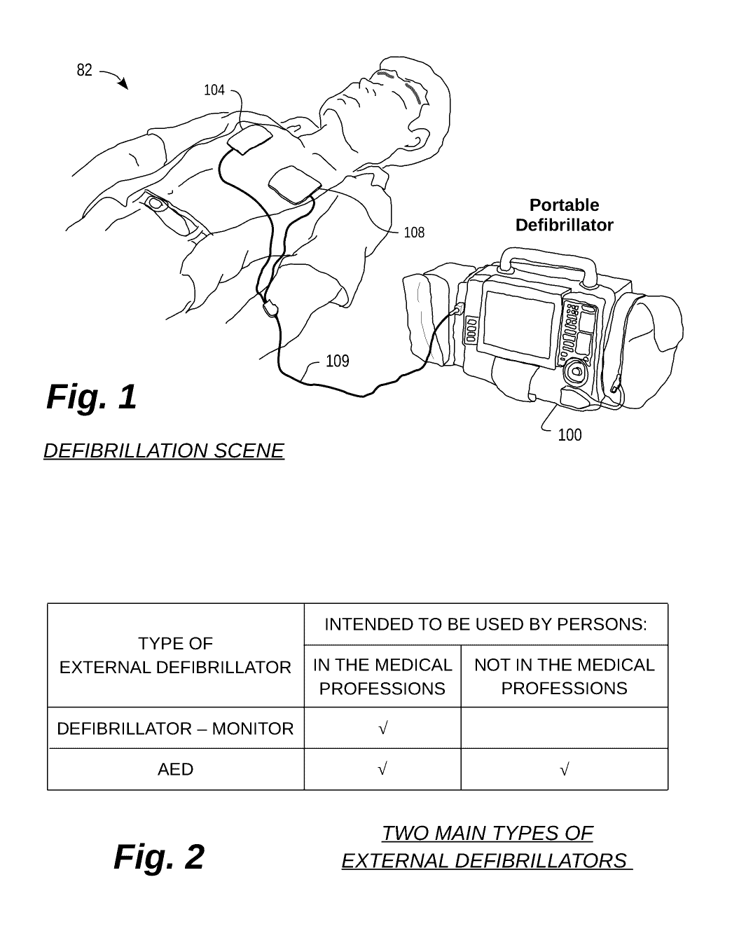

FIG. 1 is a diagram of a defibrillation scene. A person 82 is lying supine. Person 82 could be a patient in a hospital or someone found unconscious. Person 82 is experiencing a medical emergency, which could be, by way of an example, Ventricular Fibrillation (VF).

A portable external defibrillator 100 has been brought close to person 82. The portable external defibrillator can also be a hybrid monitor/defibrillator 82. At least two defibrillation electrodes 104, 108 are usually provided with external defibrillator 100. Electrodes 104, 108 are coupled with external defibrillator 100 via electrode leads 109. A rescuer (not shown) has attached electrodes 104, 108 to the skin of person 82. Defibrillator 100 is monitoring cardiac rhythms and potentially administering, via electrodes 104, 108, a brief, strong electric pulse through the body of person 82. The pulse, also known as a defibrillation shock, goes through the person's heart in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing examples of types of external defibrillators and their primary intended users. A first type of defibrillator 100 is generally called a defibrillator-monitor (or monitor-defibrillator) because it is typically formed as a single unit in combination with a patient monitor. Alternatively, the defibrillator-monitor may be a modular device with separable components. For example, in one alternative embodiment, the defibrillator-monitor may include a base component and a plurality of detachable pods. Each pod communicates with the base component, perhaps wirelessly. Certain pods may be used to collect information about a patient, such as vital statistics. One example of such an alternative system is described in U.S. Pat. No. 8,738,128 entitled “Defibrillator/Monitor System Having A Pod With Leads Capable Of Wirelessly Communicating,” the disclosure of which is hereby incorporated by reference for all purposes. A defibrillator-monitor is intended to be used by medical professionals, such as doctors, nurses, paramedics, emergency medical technicians, etc. Such a defibrillator-monitor is generally intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals, such as a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator.” An AED typically makes the shock/no shock determination by itself, automatically. It can typically sense enough physiological conditions of the person 82 using only the defibrillation electrodes 104, 108 shown in FIG. 1. An AED can either administer the shock automatically, or instruct the user to do so, e.g. by pushing a button.

There are other types of external defibrillators in addition to those listed in FIG. 2. For example, a hybrid defibrillator can have aspects of an AED and also of a defibrillator-monitor. A usual such aspect is additional ECG monitoring capability.

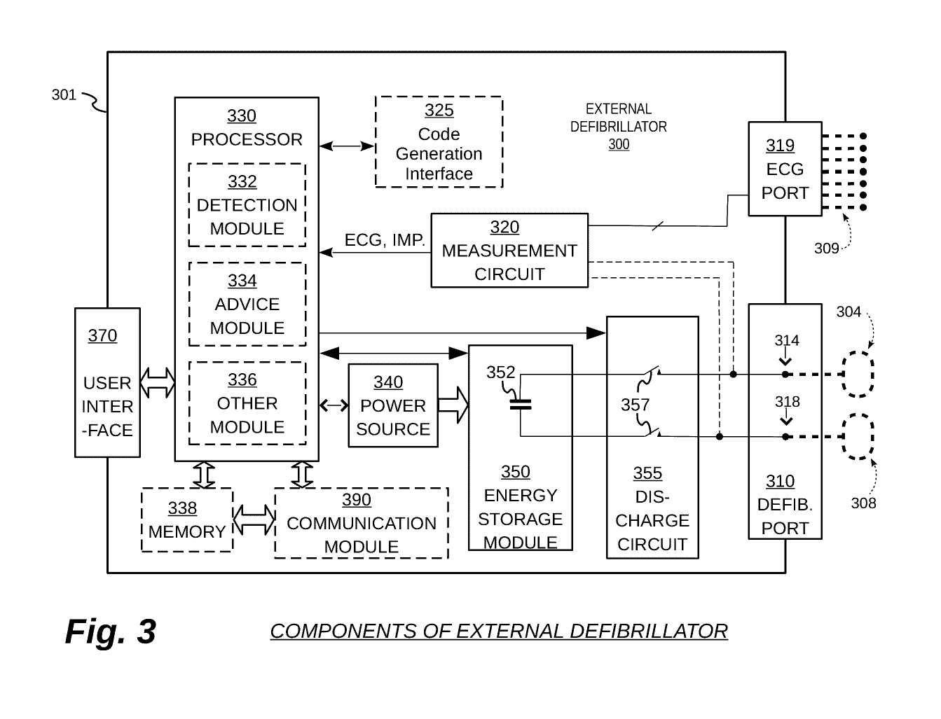

FIG. 3 is a diagram showing components of an external defibrillator 300 made according to embodiments. These components can be, for example, in external defibrillator 100 of FIG. 1. Plus, the components shown in FIG. 3 can be provided in a housing 301, also known as a casing.

External defibrillator 300 is intended for use by a user, who would be the rescuer. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding an electrical charge to person 82 via electrodes 304, 308. The electrical charge may be stored in defibrillator 300, as discussed below.

If defibrillator 300 is a defibrillator-monitor, as was described with reference to FIG. 2, it will frequently also have an ECG port 319 in housing 301, for plugging in ECG lead wires 309. ECG lead wires 309 can sense an ECG signal, such as any of the ECG lead signals that comprise a common 12-lead ECG recording. Other types of ECG lead signals are equally applicable. A defibrillator-monitor could have additional ports that are not shown.

In one embodiment, the defibrillator 300 may include a code generation component 325. In one specific implementation, the code generation component 325 includes functions and operations to interact with processor 330 (described below) to test and evaluate the status of operation of various other components of the defibrillator 300. The code generation component 325 is also configured to dynamically generate a matrix code (described below) that includes information derived from or summarizing the results of the tests and evaluations of the other components. The code generation component 325 may additionally include unique identifying information about the defibrillator 300, such as a model number and/or serial number, into the matrix code.

Defibrillator 300 also includes a measurement circuit 320. Measurement circuit 320 receives physiological signals from ECG port 319, and also from other ports, if provided. These physiological signals are sensed, and information about them is rendered by measurement circuit 320 as data, or other signals, etc.

Defibrillator 300 also includes a processor 330, which may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions too numerous to list here. In addition, if flow monitor component 325 is provided, it may be implemented as a module executing, at least in part, within processor 330.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. In addition, memory 338 can store prompts for the user, etc. Moreover, memory 338 can store patient data, such as, for example, data regarding how much fluid may have been administered to patient 82 as detected by the flow monitor component 325.

Defibrillator 300 may also include a power source 340. To enable portability of defibrillator 300, power source 340 typically includes a battery. Such a battery is typically implemented as a battery pack, which can be rechargeable or not. Sometimes, a combination of rechargeable and non-rechargeable battery packs is used. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, or the like.

Defibrillator 300 moreover includes a discharge circuit 355. Discharge circuit 355 can be controlled to permit the energy stored in module 350 to be discharged to nodes 314, 318, and thus also to defibrillation electrodes 304, 308. Discharge circuit 355 can include one or more switches 357. Those can be made in a number of ways, such as by an H-bridge, or the like.

Defibrillator 300 further includes a user interface 370. User interface 370 can be made in any number of ways. For example, interface 370 may include a screen, to display what is detected and measured, provide visual feedback to a rescuer for their resuscitation attempts, and so on. User interface 370 may also include a speaker to issue audible signals, such as voice prompts, or the like. The user interface 370 may issue prompts to the user, visually or audibly, so that the user can administer CPR, for example. Interface 370 may additionally include various controls, such as pushbuttons, keyboards, touch screens, and so on. In addition, discharge circuit 355 can be controlled by processor 330, or directly by user via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.
Embodiments of an Event-Driven System for Transmitting Treatment Data to Registered Recipients

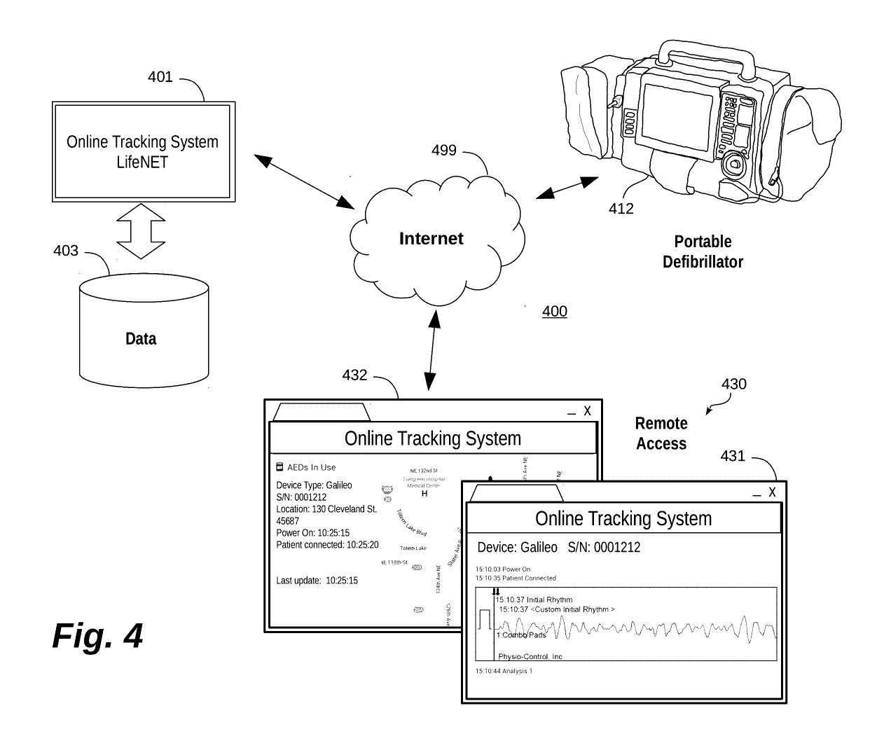

One embodiment of an event-driven system for transmitting treatment data to registered recipients is now described with reference to FIGS. 4-7. Turning first to FIG. 4, a medical system 400 is shown that implements a medical device management system 401 for managing a system of medical devices, such as a complement of portable defibrillators 412. In this embodiment, the management system 401 executes on one or more servers accessible over a wide area network 499, such as the Internet. A customer makes use of the management system 401 by registering one or more of its medical devices, such as portable defibrillators 412, with the management system 401 for receipt of treatment data. As discussed above, medical devices may be configured to record and transmit patient data and other data, such as data concerning the medical device itself. The data measured, recorded, and/or detected by the medical device is collectively referred to as “treatment data.” The treatment data is stored by the management system 401 in a data store 403.

The treatment data is accessible over the wide area network 499 by remote computing devices 430. In one specific embodiment, the remote computing devices 430 implement browsing software that retrieves web-accessible content from the management system 401 in order to visualize the treatment data. As shown, the treatment data may include patient data 431, such as electrocardiogram waveforms and/or other information. The treatment data may also include information about the medical device independent of a patient, such as the geographic location 432 of the medical device, or the like.

In operation, when the medical device 412 is activated (e.g. a cabinet is opened, the medical device turns on, electrodes are placed on a patient, or similar), AED events, waveforms, and other AED data is transmitted to the management system 401. That data is stored in data store 403 for any use that may be made with that data locally. In addition, the treatment data may also be transmitted to any recipients that have registered to receive treatment data. In this way, the treatment data may be sent to various computing devices, including mobile computing devices, for viewing and/or assessment.

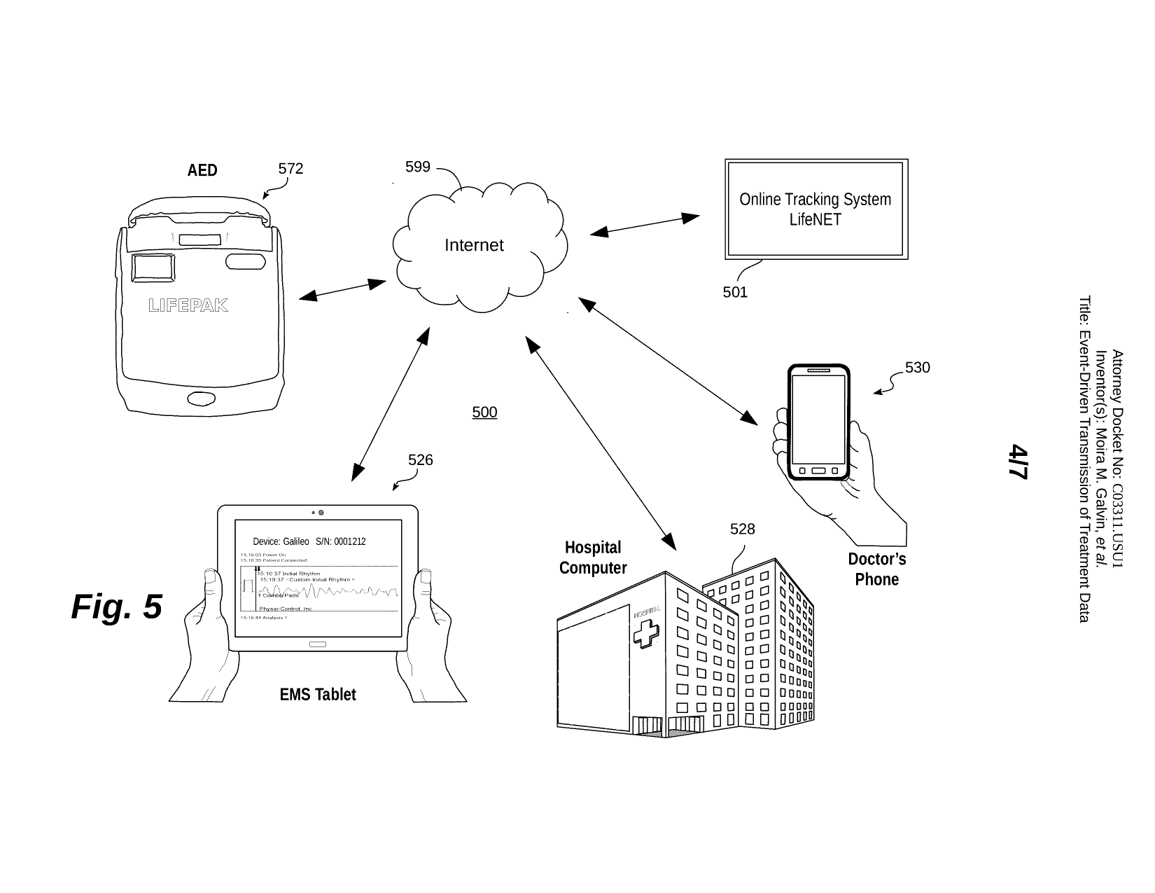

Turning to FIG. 5, a conceptual illustration of an event-driven system 500 for transmitting treatment data to recipients is shown. As illustrated, a medical device 512 transmits treatment data to a management system 501. A number of possible recipients are also shown. Recipients that register to receive treatment data receive that data upon the occurrence of specific events. Examples of potential recipients that may register to receive treatment data include local emergency response teams 526, an admitting hospital 528 that will receive the patient, and a doctor 530 who is or will be providing care for the patient.

Generally speaking, upon the occurrence of a first event, the medical device 512 initiates a communication session with the management system 501 over a wide area network 599. In this embodiment, the communication session is a secure encrypted communication link between the medical device and the management system 501. In one particular implementation, the secure sockets layer protocol or the transport layer security protocol may be used to encrypt communication between sending and receiving device. In this particular embodiment, once initiated the communication session remains open and active for the duration of the medical incident until treatment terminates.

Using the system described herein, medical incident information collected by the medical device 512 is available to local emergency response teams 526 for activating AED use protocols. Medical incident treatment data (e.g, rhythm, potentially the rhythm category, CPR performance, shocks delivered, joule escalation) is available to EMS crew that will be on scene as soon as possible. Important treatment data (e.g, first rhythm, CPR performance, shocks delivered, joule escalation) is also available to admitting hospital prior to arrival, which enhances the quality of treatment that can be immediately provided at the hospital 528. In addition, important treatment data can be delivered directly to the treating physician 530, such as on a smartphone or other ultra-compact mobile computing device.

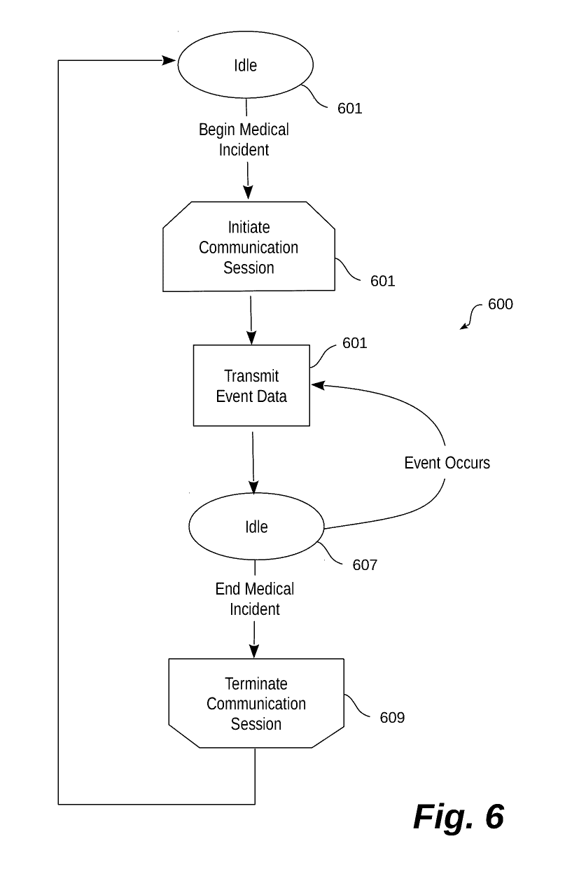

Turning now to FIG. 6, a functional flow diagram generally illustrates one embodiment of a process performed by an illustrative system for transmitting treatment data. The system begins at an idle state 601 where a medical device may be deployed in the field but not in use. As is common, portable defibrillators may be installed at various locations in public places for immediate use in medical emergencies. When a medical incident occurs, the medical device initiates a communication session (603) with a remote management system.

As noted above, the communication session may be established using built-in wireless network access. Notice of the medical incident may take many forms, but is generally characterized by an event that indicates the medical device is being put to use. Examples of such an event include the opening of a cabinet in which the medical device is stored, powering on the medical device, detaching components from the medical device (e.g., electrode leads), attaching components of the medical device (e.g., electrode leads) to a patient, or the like. In one embodiment, the communication session is initiated automatically without any need for user intervention. Eliminating the need for a user to remember to initiate the communication session or helps to eliminate one possible mistake.

With a communication session open, the medical device begins transmitting medical incident treatment data (605) upon the occurrence of an event. Again, in one preferred embodiment, transmission of the treatment data begins without user intervention and without requiring the user to activate any reporting feature. Very many events may generate treatment data. For example, the types of events that may generate treatment data include powering on the medical device, a power-on self test, attachment of electrode leads to a patient, beginning to record a heart rhythm, application of a defibrillation shock, the occurrence of ROSC, or the like. The treatment data that may be transmitted is extensive, and includes waveforms recorded during the treatment, geographic data about the location of the medical device, sound and/or video captured at the scene, and the like. The treatment data (or portions of it) is transmitted to network accessible servers and distributed to various destinations based on individual need. More specifically, as noted above, certain service providers may only need or have interest in certain portions of the treatment data. In other words, first responders may have a greater need to know the geographic location of the medical device, while an attending hospital is only interested in the heart rhythm waveforms. Each recipient registers to receive any data in which the recipient is interested.

The system may also incorporate optional features that allow scene audio and video data to be transmitted to 911 dispatch centers, which in turn can provide real-time responder coaching based on event information and audio and visual information.

In this embodiment, particular treatment data is transmitted in response to the occurrence of each event as noted above. Accordingly, the system may go in to a temporary idle state (607) awaiting the occurrence of another event for which treatment data should be transmitted. When such an event occurs, the process transmits that data (605) using the established communication session, and again returns to idle (607) awaiting the occurrence of the next event.

Once the medical incident has concluded, and there are no more events for which treatment data should be transmitted, the medical device terminates the communication session 609. At this point, the system returns to the idle state and awaits another medical incident. It should be noted that final post-event data may also be transmitted before the communication session is terminated. For example, a power-down event for the medical device may result in a final set of treatment data to be transmitted for post-event review.

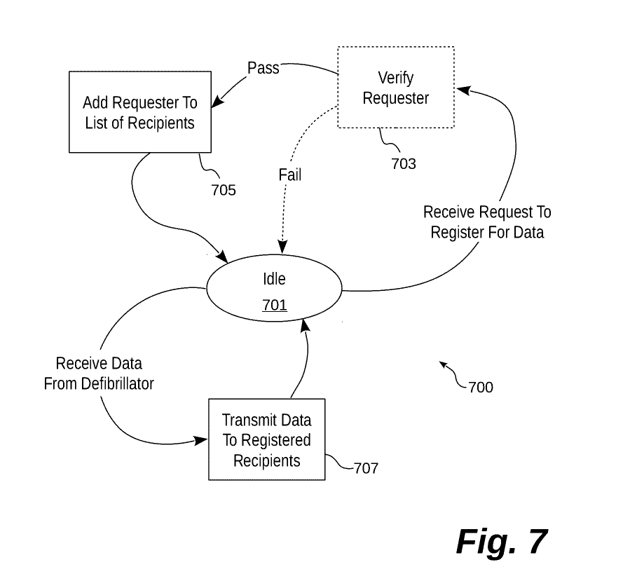

Turning now to FIG. 7, a functional flow diagram generally illustrates one embodiment of a process 700 performed by an illustrative system for registering recipients to receive treatment data based on events for which that recipient is interested. The process 700 begins with the system in an idle condition (701) which represents a medical system operating normally. While in the idle condition, the system may receive a request to register for treatment data from a potential recipient. Potential recipients may include any number of individuals or entities interested in treatment data. As discussed above, potential recipients may be emergency response personnel notified that a medical incident has occurred, a physician who will be treating the patient, or a hospital that expects to receive the patient.

When a request is received from a recipient to register for treatment data, the process may perform an optional verification step (703) to ensure that a particular recipient is authorized to receive treatment data. The verification may take any one of many forms, such as confirming certain login credentials, confirming an originating address for the intended recipient, or the like. If the verification fails, the system returns to idle.

If the optional verification is affirmative, the recipient is added to a list of recipients to receive treatment data (705). In this particular embodiment, the recipient is associated with particular events. A medical management system that receives treatment data maintains a list of recipients that may be dynamically generated for each medical incident, or it may be a persistent list of recipients registered to receive notifications for any medical devices that begin transmitting treatment data based on the registered events. Once the recipient is registered to receive treatment data, the process returns to the idle state 701.

At some point, the system may begin receiving treatment data due to the occurrence of a particular event associated with a medical incident. When that happens, the system may store that information and it also begins transmitting the treatment data (or select portions thereof) to any recipients registered to receive that particular treatment data (707). For example, once a medical incident has begun, an emergency response team may select to receive (or have been registered to receive) geographic location information to assist in locating the patient. In another example, the emergency response team may register to receive geographic location information about medical incidents in advance of any such incidents occurring.

Similarly, heart rhythm waveforms may be transmitted to an attending hospital so that hospital staff may begin preparations to treat the patient. In this way, the treatment data is made available most quickly to those individuals most in need of that data to treat the patient. In certain embodiments, hospitals may register in advance to receive treatment data based on medical incidents that occur in a certain geographical location, which are of a certain type, or the like. Once the relevant treatment data is transmitted to the registered recipients, the system returns to the idle condition 701 and awaits further treatment data.

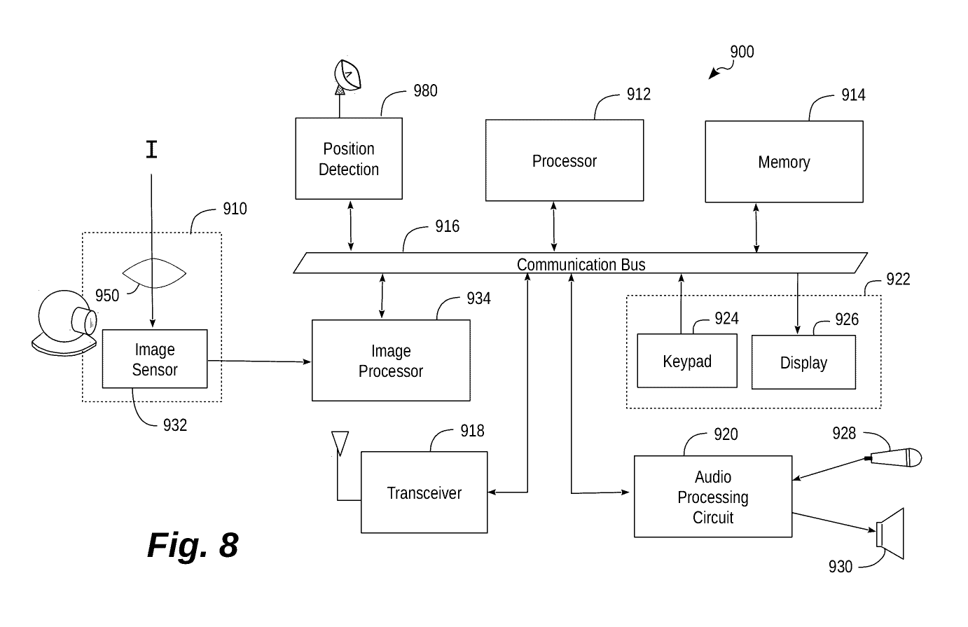

FIG. 8 is a functional block diagram generally illustrating one possible example of a computing device 900 that may be used in various embodiments. As shown, computing device 900 includes several functional components to enable scanning matrix codes and transmitting information. The example computing device may be implemented as one or more of any number of portable devices, such as a mobile phone, a tablet computer, a personal digital assistant, a notebook or ultrabook computer, or the like. Alternatively, the computing device could be implemented as a component in a location-aware cabinet or other housing for a portable medical device. For example, the example computing device could be implemented as an AED containment bag including a camera and electronics, or as a cabinet in which an AED or other medical device should be stored.

The computing device 900 comprises a processor 912, a memory 914, communication circuit 916, transceiver 918, audio processing circuit 920, user interface 922, image sensor 932, image processor 934, and optical system 950. Microprocessor 912 controls the operation of the computing device 900 according to programs stored in program memory 914. The communication circuit 916 interfaces the microprocessor 912 with the various other components, such as the user interface 922, transceiver 918, audio processing circuit 920, and image processing circuit 934. User interface 922 may include a keypad 924 and a display 926. Keypad 924 allows the operator to key in alphanumeric characters, enter commands, and select options. The display 926 allows the operator to view output data, such as entered information, output of the computing device 900, images or other media, and other service information. In certain computing devices, the user interface 922 combines the keypad 924 and the display 926 into a touchpad display.

The computing device 900 also includes a microphone 928 and speaker 930 though certain mobile communication devices, such as a PDA or palm-top computer may not have such features. Microphone 928 converts sounds into electrical audio signals, and speaker 930 converts audio signals into audible sound. Audio processing circuit 920 provides basic analog output signals to the speaker 930 and accepts analog audio inputs from the microphone 928. Transceiver 918 is coupled to an antenna 936 for receiving and transmitting signals on a suitable communications network (not shown).

Image sensor 932 captures images formed by light impacting on the surface of the image sensor 932. The image sensor 932 may be any conventional image sensor 932, such as a charge-coupled device (CCD) or complementary metal oxide semiconductor (CMOS) image sensor. Additionally, the image sensor 932 may be embodied in the form of a modular camera assembly with or without an integrated optical system 950. Image processor 934 processes raw image data collected by the image sensor 932 for subsequent output to the display 926, storage in memory 914, or for transmission by the transceiver 918. The image processor 934 is a conventional signal microprocessor programmed to process image data, which is well known in the art. A position sensor 980 detects the position of the computing device 900 and generates a position signal that is input to the microprocessor 912. The position sensor 980 may be a Global Positioning System sensor, potentiometer, or other measuring device known in the art of electronics. Alternatively, the position sensor 980 may be omitted and the position of the computing device 900 may be determined programmatically, such as by collecting information about the computing device (e.g., network address(es), network identifiers, wireless signal strength, and the like) and resolving that information into a geographic location, perhaps through the use of a remote service.

Other embodiments may include combinations and sub-combinations of features described or shown in FIGS. 3-9, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

In the foregoing description, numerous details have been set forth in order to provide a sufficient understanding of the described embodiments. In other instances, well-known features have been omitted or simplified to not unnecessarily obscure the description.

A person skilled in the art in view of this description will be able to practice the disclosed invention. The specific embodiments disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems. The following claims define certain combinations and subcombinations of elements, features, steps, and/or functions, which are regarded as novel and non-obvious. Additional claims for other combinations and subcombinations may be presented in this or a related document.

What is claimed is:

1. A medical device system, comprising:
a plurality of medical devices, each medical device including a communication component and a data collection component, the communication component being configured for secure communications over a network communication session, each medical device being further configured to monitor for the occurrence of particular events during a treatment incident, each medical device being further configured to transmit treatment data associated with the particular events during the treatment incident; and
a management component accessible over a wide area network, the management component maintaining a list of registered recipients that have requested to be provided treatment data based on the occurrence of particular events during the treatment incident,
wherein at least one medical device is configured to initiate a secure communication session with the management component in response to an initial event during the treatment incident and to begin transmitting treatment data over the secure communication session for the duration of the treatment incident, and further wherein the management component transmits treatment data to any recipients on the list of recipients that have registered to receive that treatment data.

2. The medical device system recited in claim 1, wherein the plurality of medical devices comprises at least one portable defibrillator.

3. The medical device system recited in claim 1, wherein the list of registered recipients includes information that identifies at least emergency response personnel, or a physician, or a hospital, or any combination of the foregoing.

4. The medical device system recited in claim 1, wherein the treatment incident involves a patient experiencing a medical condition, and wherein the treatment data comprises information about the patient undergoing treatment for the medical condition.

5. The medical device system recited in claim 4, wherein the information about the patient includes heart rhythm waveform data.

6. The medical device system recited in claim 1, wherein the treatment data comprises information sufficient to identify a geographic location for the at least one medical device.

7. The medical device system recited in claim 6, wherein the at least one medical device includes a location identification component operative to determine the geographic location of the at least one medical device.

8. The medical device system recited in claim 6, wherein the geographic location information about the at least one medical device is maintained by the management component.

9. The medical device system recited in claim 1, wherein the at least one medical device is further configured to terminate the secure communication session at the conclusion of the treatment incident.
